(19) United States
(12) Patent Application Publication
(10) Pub. No.: US 2008/0032961 A1
(43) Pub. Date: Feb. 7, 2008

(54) Therapy employing LXRalpha modolators

(76) Inventors: Krister Bamberg, Molndal (SE); Hilde Nebb, Blindern (NO)

Correspondence Address: WHITE & CASE LLP;PATENT DEPARTMENT, 1155 AVENUE OF THE AMERICAS, NEW YORK, NY, 10036, US

(21) Appl. No.: 11/672,709

(22) Filed: Feb. 8, 2007

Related U.S. Application Data
(63) Continuation of application No. 10/466,333, filed on Nov. 10, 2003, now abandoned.

(30) Foreign Application Priority Data
Jan. 25, 2001 (GB) 0101933.0

(51) Int. Cl. A61K 31/4436; A61K 31/427; A61K 31/56; A61P 3/06; A61P 3/10; C12N 5/06

(52) U.S. Cl. 514/182; 435/377; 514/342; 514/369; 514/789

(57) ABSTRACT

The invention also relates to the use of active modulators of LXRα activity or expression in stimulation of pre-adipocyte differentiation and hence also in the treatment of insulin resistance syndrome, or dyslipidemia, or type 2 diabetes.

### FIELD OF THE INVENTION

The invention relates to methods of screening test compounds for their ability to stimulate pre-adipocyte differentiation by measuring their activity as a modulator of LXRα activity or expression. The invention also relates to the use of active modulators of LXRα activity or expression in stimulation of pre-adipocyte differentiation and hence also in the treatment of insulin resistance syndrome, or dyslipidemia, or type 2 diabetes.

### BACKGROUND OF THE INVENTION

PPARγ is an established master switch for driving adipocyte differentiation. Retrovirus-mediated expression of PPARγ in a fibroblast cell line (NIH-3T3) conferred an adipocyte phenotype onto this otherwise non-adipogenic cell (Tontonoz et al., 1994). Treatment of 3T3-L1 pre-adipocytes with Pioglitazone (a PPARγ agonist of the thiazolidinedione class) enhanced the insulin or insulin-like growth factor-I (IGF-I)-regulated differentiation as monitored by the rate of lipogenesis or triglyceride accumulation (Kletzien et al., 1992). Pioglitazone caused both a leftward shift and enhanced maximum response for the IGF-I-regulated differentiation of the cells, consistent with the idea that the drug enhances the sensitivity of cells to polypeptide hormones. PPARγ agonists are therefore promoters of adipocyte differentiation and insulin sensitisers and are prescribed clinically to treat type 2 diabetes.

Here we show that a thiazolidinedione, Darglitazone, leads to increased expression of the nuclear receptor LXRα in 3T3-L1 adipocytes and in human primary adipocytes. In addition we show that activation of LXRα leads to differentiation of pre-adipocyte cells to adipocytes.

The LXRs were first identified as orphan members of the nuclear receptor superfamily (Willy et al., 1995) and have later been shown to be activated by a specific class of naturally occurring, oxidised derivatives of cholesterol, including 22(R)-hydroxycholesterol, 24(S)-hydroxycholesterol, and 24,25(S)-epoxycholesterol (Janowski et al., 1996, Janowski et al., 1999). Two members of the LXR family have been identified: the tissue restricted (mainly liver, intestine, kidney and adipocytes) LXRα and the ubiquitous LXRβ (Peet et al., 1998, Repa & Mangelsdorf, 1999). When cholesterol is in excess and its oxidised metabolites are present, LXRα is activated and induces transcription of the Cyp7a1 gene, which encodes the rate-limiting enzyme in the classical bile acid synthesis pathway cholesterol 7α-hydroxylase. Upregulation of cholesterol 7α-hydroxylase enhances conversion of cholesterol to bile acids, thereby reducing the amount of circulating cholesterol. The role of LXRα as a key regulator of cholesterol homeostasis has been studied in mice homozygous for a disrupted LXRα gene. These genetically modified mice are apparently healthy and fertile when fed with a normal diet. However, when given a high content cholesterol diet (0,2% or 2%), hepatomegaly with cholesterol accumulation occurs, leading to hepatic failure, and also failure of Cyp7a1 transcription induction was detected. These results provide evidence that LXRα is required to regulate Cyp7a1 expression in mice and that this is very important for maintenance of cholesterol homeostasis. These observations have led to the suggested use of LXRα agonists to increase the synthesis of bile acids as a means to lower the level of blood cholesterol.

Recently the gene encoding the ATP-binding cassette transporter protein 1 (ABC-1), was reported to be transcriptionally regulated by LXRα (Costet et al., 2000, Repa et al., 2000). The ABC-1 transporter is involved in cellular efflux of cholesterol to high density lipoproteins (HDL). Interestingly, several genetic defects in this transporter are also characterised by accumulation of cholesterol in various tissues and increased risk of coronary artery disease in patients belonging to a Tangiers disease cohort. This indicates that LXRα may have additional roles in the regulation of cholesterol levels besides controlling the Cyp7a1 gene.

WO 93/06215 (EP609240), The Salk Institute. This application describes the cloning of five new orphan receptors belonging to the steroid/thyroid superfamily of receptors, one (designated XR2) has later been shown to be the human LXRα.

WO 96/21726, The Salk Institute. This application describes the characterisation of LXRα and claims certain response elements, LXR/RXR heterodimers, and LXR based assays.

WO 99/18124 (EP1021462) Merck & Co. This application covers methods for identifying agonist and antagonists of nuclear receptors. The claimed methods comprises the use of a nuclear receptor or a ligand binding domain thereof labelled with a first fluorescent reagent; a nuclear receptor co-activator or a binding portion thereof labelled with a second fluorescent reagent; and measuring FRET between the first and second fluorescent reagents. LXR is exemplified as one of the nuclear receptors of the claimed methods and SRC-1 as a co-activator.

WO 00/34461 University of Texas. This application covers various aspects of modulating cholesterol metabolism, such as LXRα knock-out mice and their use in screens, screen for LXRα agonists for their ability to increase bile acid synthesis, screening for substances reducing cholesterol levels or increasing bile acid synthesis using LXRα knock-outs, screen for modulators of ABC1 expression.

### SUMMARY OF THE INVENTION

The present invention is based on the discovery that agonists of LXRα activity stimulate differentiation of pre-adipocytes. In addition, differentiation of a pre-adipocytes is accompanied by an increased expression of LXRα. Stimulation of differentiation of pre-adipocytes is useful also in the treatment of insulin resistance syndrome, or dyslipidemia, or type 2 diabetes.

In one aspect, the invention features a method of stimulating pre-adipocyte differentiation in a cell comprising administering a LXRα agonist to a cell, wherein the agonist stimulates pre-adipocyte differentiation. In one embodiment, the cell is a mammalian cell such as an adipocyte cell, a 3T3-L1 pre-adipocyte cell, or a 3T3-L1 adipocyte cell. In one embodiment, the LXRα agonist is an oxidized derivative of cholesterol such as 22(R)-hydroxycholesterol, 24(S)-hydroxycholesterol, and 24,25(S)-epoxycholesterol. In another embodiment, the LXRα agonist is a thiazolidinedione compound such as darglitazone, rosiglitazone, pioglitazone, or troglitazone, and their pharmaceutically acceptable salts.

In another aspect, the invention features a method of treating a disorder associated with aberrant pre-adipocyte differentiation. The method includes administering a therapeutically effective amount of a LXRα modulator to a mammal, wherein the LXRα modulator stimulates pre-adipocyte differentiation. In one embodiment the LXRα modulator is an oxidized derivative of cholesterol such as 22(R)-hydroxycholesterol, 24(S)-hydroxycholesterol, and 24,25(S)-epoxycholesterol. In another embodiment, the LXRα modulator is a thiazolidinedione compound such as darglitazone, rosiglitazone, pioglitazone, or troglitazone, and their pharmaceutically acceptable salts. The disorder can be any disorder which has an aberrant adipocyte differentiation, e.g., the disorder can be insulin resistance syndrome, dyslipidemia or type 2 diabetes. The LXRα modulator can be administered in any manner known in the art including orally, topically, intravenously, transdermally, rectally, or parentally. In one embodiment the modulator is administered to the mammal in a pharmaceutical composition comprising a pharmaceutically acceptable carrier or excipient.

In another aspect the invention features a method of increasing the level of LXRα expression or activity, comprising administering a pharmaceutically effective amount of a LXRα modulator. In one embodiment the LXRα modulator is an oxidized derivative of cholesterol such as 22(R)-hydroxycholesterol, 24(S)-hydroxycholesterol, and 24,25(S)-epoxycholesterol. In another embodiment, the LXRα modulator is a thiazolidinedione compound such as darglitazone, rosiglitazone, pioglitazone, or troglitazone, and their pharmaceutically acceptable salts In one embodiment the modulator is administered to a pre-adipocyte cell in a mammal. In another embodiment the mammal has insulin resistance syndrome, dyslipidemia or type 2 diabetes.

The invention further relates to the use of a variety of procedures for using the LXRα receptor in the discovery of modulators of the receptor function or expression, such modulators may be used in stimulating pre-adipocyte differentiation and therefore used to modify or ameliorate insulin resistance syndrome or dyslipidemia or type 2 diabetes.

In one aspect, the invention features a method for identifying a compound that stimulates pre-adipocyte differentiation. The method includes providing a cell comprising a LXRα regulatory sequence operatively linked to a reporter gene; introducing a test compound into the cell; assaying for transcription of the reporter gene in the cell, wherein an increase in transcription in the presence of the compound compared to transcription in the absence of the compound indicates that the compound stimulates pre-adipocyte differentiation. The cell can be any cell such as a mammalian cell. In one embodiment the cell is an adipocyte cell, a 3T3-L1 pre-adipocyte cell, or a 3T3-L1 adipocyte cell. The reporter gene can encode a luciferase, a chloramphenicol acetyl transferase, a beta-galactosidase, an alkaline phosphate, or a fluorescent protein.

In another aspect, the invention features a method of identifying a compound which binds to a LXRα polypeptide comprising contacting a LXRα polypeptide, or a cell expressing a LXRα polypeptide, with a test compound; and determining if the polypeptide binds to the test compound. The binding of the test compound to the polypeptide can be detected by direct detecting of the compound to the polypeptide or by a competition binding assay.

The invention further features a method for identifying a compound which modulates the activity of a LXRα polypeptide comprising contacting a LXRα polypeptide with a test compound and assaying for the ability of the test compound to stimulate pre-adipocyte differentiation, wherein an increase in the ability of the polypeptide to stimulate pre-adipocyte differentiation indicates that the compound modulates the activity of the LXRα polypeptide.

In another aspect, the invention features a method of identifying an agonist of LXRα which includes contacting a LXRα protein, or fragment thereof, a LXRα coactivator and a compound; and determining if the LXRα protein, or fragment thereof, and the LXRα coactivator interact, wherein an interaction between the LXRα protein, or fragment thereof, and the LXRα coactivator indicates that the compound is a LXRα agonist. In one embodiment the LXRα co-activator is a steroid receptor co-activator.

In yet another aspect, the invention features a method of identifying an agonist of LXRα which includes contacting a LXRα protein, or fragment thereof, a LXRα heterodimerization partner or fragment thereof, and a compound; and determining if the LXRα protein, or fragment thereof, and the LXRα heterodimerization partner, or fragment thereof, interact, wherein an interaction between the LXRα protein, or fragment thereof, and the LXRα heterodimerization partner, or fragment thereof, indicates that the compound is a LXRα agonist. In one embodiment, the LXRα heterodimerization partner is a retinoid X receptor.

The invention relates to pharmaceutical compositions containing such a modulator discovered by the methods described in this application and the use of the modulator or pharmaceutical composition comprising such modulator in stimulating pre-adipocyte differentiation and therefore used to modify or ameliorate insulin resistance syndrome or dyslipidemia or type 2 diabetes.

In one aspect the invention feature the use of a LXRα modulator in the manufacture of a medicament for the treatment of a disorder associated with aberrant pre-adipocyte differentiation, wherein the LXRα modulator stimulates pre-adipocyte differentiation. In one embodiment the LXRα modulator is an oxidized derivative of cholesterol such as 22(R)-hydroxycholesterol, 24(S)-hydroxycholesterol, and 24,25(S)-epoxycholesterol. In another embodiment the LXRα modulator is a thiazolidinedione compound such as darglitazone, rosiglitazone, pioglitazone, or troglitazone, and their pharmaceutically acceptable salts. The disorder can be any disorder associated with aberrant pre-adipocyte differentiation such as insulin resistance syndrome, dyslipidemia or type 2 diabetes. The LXRα modulator can be administered orally, topically, intravenously, transdermally, rectally, or parentally.

In yet another aspect the invention features a pharmaceutical formulation for use in the treatment of a disorder associated with aberrant pre-adipocyte differentiation. In one embodiment the LXRα modulator is an oxidized derivative of cholesterol such as 22(R)-hydroxycholesterol, 24(S)-hydroxycholesterol, and 24,25(S)-epoxycholesterol. In another embodiment the LXRα modulator is a thiazolidinedione compound such as darglitazone, rosiglitazone, pioglitazone, or troglitazone, and their pharmaceutically acceptable salts. The disorder can be any disorder associated with aberrant pre-adipocyte differentiation such as insulin resistance syndrome, dyslipidemia or type 2 diabetes. The LXRα modulator can be administered orally, topically, intravenously, transdermally, rectally, or parentally.

### BRIEF DESCRIPTION OF THE FIGURES

FIG. 1 depicts a bar chart showing Northern blot analysis of total RNA isolated from differentiated 3T3-L1 cells probed with an LXRα probe.

FIG. 2 depicts a bar chart showing Northern blot analysis of 20 μg total RNA obtained from fully differentiated 3T3-L1 cells stimulated with 22-R (5 μM), Darglitazone (1 μM) alone or in combination for 24 hours, probed with an LXRα probe.

FIG. 3 depicts a bar chart showing Northern blot analysis of polyA+ RNA isolated from human adipocytes grown in the presence of 1 μM Darglitazone (Dar), 5 μM 22-R-hydroxy cholesterol (22-R—OH) or both (Dar+22-R—OH), probed with an LXRα probe.

### DETAILED DESCRIPTION OF THE INVENTION

Treatment of 3T3-L1 pre-adipocytes with an LXRα agonist, 22-R-hydroxycholesterol, enhances adipocyte differentiation of 3T3-L1 pre-adipocytes. This demonstrates that LXRα not only is very important for maintenance of cholesterol homeostasis but represents an important regulatory factor in adipocyte differentiation. Treatment with an LXRα modulator of activity or expression can lead to stimulation of pre-adipocyte differentiation and have utility in improving insulin sensitisation and therefore constitutes a novel treatment for dyslipidemia and insulin resistance and type 2 diabetes.

This invention provides a method for stimulation of pre-adipocyte differentiation comprising the administration of an effective amount of a modulator of the activity or expression of LXRα to a patient in need of such treatment.

Modulation, preferably by an “upregulator”) of the expression of LXRα by a compound may be brought about, for example, through altered gene expression levels or message stability. Modulation, preferably by an “agonist”, of the activity of LXRα by a compound may be brought about for example through compound binding to LXRα, LXRα/RXRα heterodimer, LXRα/co-activator or LXRα/RXRα/co-activator complexes.

In a further aspect of the present invention we provide a method for the provision of an adipocyte differentiation agent, which method comprises using one or more putative modulator of LXRα expression or activity as test compounds in one or more procedure to measure the ability of the test compound to modulate LXRα, and selecting an active compound for use as an agent able to stimulate pre-adipocyte differentiation.

Convenient test procedures include the use of animal models to test the role of the test compound. These will typically involve the administration of compounds by intra peritoneal injection, subcutaneous injection, intravenous injection, oral gavage or direct injection via canullae into the blood stream of experimental animals. The effects on insulin sensitivity, lipid profiles, food intake, body temperature, metabolic rate, behavioural activities and body weight changes may all be measured using standard procedures.

Suitable modulators may be firstly identified by screening against the isolated LXRα receptor or fragment or chimeirc form thereof.

Preferably the screen is selected from:
i) measurement of LXRα activity using a reporter gene assay comprising a cell line which expresses LXRα and a reporter gene coupled to an LXRα response element and assaying for expression of the reporter gene.
ii) measurement of LXRα activity using purified LXRα protein or a fragment thereof and a co-activator or a fragment thereof, and assaying the interaction between LXRα and the co-activator, preferably by time resolved fluorescence resonance energy transfer or by scintillation proximity assay.
iii) measurement of LXRα activity using purified LXRα protein or a fragment thereof and a heterodimerization partner or a fragment thereof, and assaying the interaction between LXRα and the heterodimerization partner, preferably by time resolved fluorescence resonance energy transfer or by scintillation proximity assay
iv) measurement of LXRα transcription or translation in a cell line expressing LXRα.
v) measurement of direct compound binding or competitive binding to LXRα, preferably by time resolved fluorescence resonance energy transfer or scintillation proximity assay.

Examples of a suitable assays can be found in WO 99/18124 (EP1021462) Merck & Co.

Examples of suitable co-activators, but not limited to, are the Steroid Receptor Coactivators, such as SRC-1, SRC-2, and SRC-3, the Nuclear Receptor CoActivators, such as NcoA-1, NcoA-2, the CREB Binding protein (CBP), p300, p/CIP, TIF-1, TIF-2, TRIP-1, and GRIP-1.

Suitable heterodimerization partners are the Retinoid X Receptors (RXR), such as RXRα, RXRβ and RXRγ, preferably RXRα.

Preferably the cell line is a 3T3-L1 pre-adipocyte cell or a 3T3-L1 adipocyte cell or any other commonly used mammalian cell line.

The mammalian LXRα receptors may be conveniently isolated from commercially available RNA, brain cDNA libraries, genomic DNA, or genomic DNA libraries using conventional molecular biology techniques such as library screening and/or Polymerase Chain Reaction (PCR). These techniques are extensively detailed in Molecular Cloning—A Laboratory Manual, 2^{nd} edition, Sambrook, Fritsch & Maniatis, Cold Spring Harbor Press.

The resulting cDNA's encoding mammalian LXRα receptors are then cloned into commercially available mammalian expression vectors such as the pcDNA3 series (InVitrogen Ltd etc. see below). An alternative mammalian expression vector is disclosed by Davies et al., J of Pharmacol & Toxicol. Methods, 33, 153-158. Standard transfection technologies are used to introduce these DNA's into commonly available cultured, mammalian cell lines such as CHO, HEK293, HeLa and clonal derivatives expressing the receptors are isolated. An alternative expression system is the MEL cell expression system claimed in our UK patent no. 2251622.

Application of a natural ligand to these cells causes activation of the transfected receptor that may cause changes in the levels of endogenous molecules such as ABC-1 or aFABP These may all be measured using standard published procedures and commercially available reagents. In addition, these cDNA's may be transfected into derivatives of these cells lines that have previously been transfected with a “reporter” gene. Examples of suitable reporter genes are esterase, phosphatases, proteases, fluorescent proteins, such as GFP, YFP, BFP, and CFP, luciferase, chloramphenicol acetyl transferase, β-galactosidase, β-glucuronidase that will “report” these intracellular changes.

These transfected cell lines may be used to identify low molecular weight compounds that activate these receptors, these are defined as “agonists”.

In addition or alternatively, the same assays can be used to identify low molecular weight compounds that antagonise the activation effect of a LXRα ligand, these are defined as “antagonists”. Antagonist may have utility in treating obesity, dyslipidemia, insulin resistance syndrome and type 2 diabetes.

The test compound may be a polypeptide of equal to or greater than, 2 amino acids such as up to 6 amino acids, up to 10 or 12 amino acids, up to 20 amino acids or greater than 20-amino acids such as up to 50 amino acids. For drug screening purposes, preferred compounds are chemical compounds of low molecular weight and potential therapeutic agents. They are for example of less than about 1000 Daltons, such as less than 800, 600 or 400 Daltons in weight. If desired the test compound may be a member of a chemical library. This may comprise any convenient number of individual members, for example tens to hundreds to thousands to millions etc., of suitable compounds, for example peptides, peptoids and other bligomeric compounds (cyclic or linear), and template-based smaller molecules, for example benzodiazepines, hydantoins, biaryls, carbocyclic and polycyclic compounds (eg. naphthalenes, pheriothiazines, acridines, steroids etc.), carbohydrate and amino acids derivatives, dihydropyridines, benzhydryls and heterocycles (eg. triazines, indoles, thiazolidines etc.). The numbers quoted and the types of compounds listed are illustrative, but not limiting. Preferred chemical libraries comprise chemical compounds of low molecular weight and potential therapeutic agents.

In a further aspect of the invention we provide the use of a modulator of LXRα receptor activity or expression as an agent able to stimulate pre-adipocyte differentiation and thereby modify or ameliorate insulin resistance syndrome or dyslipidemia or type 2 diabetes.

In a further aspect of the present invention we provide a method of treating insulin resistance syndrome, dyslipidemia or type 2 diabetes which method comprises administering to a patient suffering such a disease a pharmaceutically effective amount of an agent, preferably identified using one or more of the methods of this invention, able to stimulate pre-adipocyte differentiation by modulating LXRα activity or expression and thereby modify or ameliorate the insulin resistance syndrome, dyslipidaemia or type 2 diabetes disease.

This invention further provides use of an agent able to stimulate pre-adipocyte differentiation by modulating LXRα activity in preparation of a medicament for the treatment of dyslipidemia or IRS or type 2 diabetes. Preferably the compound is an LXRα agonist.

According to another aspect of the present invention there is provided a method of preparing a pharmaceutical composition which comprises:

i) identifying an agent as useful for stimulation of pre-adipocyte differentiation according to a method as described herein; and

ii) mixing the agent or a pharmaceutically acceptable salt thereof with a pharmaceutically acceptable excipient or diluent.

It will be appreciated that the present invention includes the use of orthologues and homologues of the human LXRα receptor.

The degree of pre-adipocyte differentiation required for the treatment of the type 2 diabetes, IRS or dyslipidemia can be almost any level of stimulation over basal levels as measured in the patient suffering from the particular disease, preferably at least 10% increase in rate over basal levels. Preferably a compound should be administered which has an affinity (Kₘ) for LXRα below 100 μM preferably below 1 μM, as measured against the isolated receptor.

The pharmaceutical composition can further comprise a PPARγ agonist, preferably a thiazolidinedione such as Darglitazone, Rosiglitazone, Pioglitazone, or Troglitazone.

By the term “orthologue” we mean the functionally equivalent receptor in other species.

By the term “homologue” we mean a substantially similar and/or related receptor in the same or a different species.

For either of the above definitions we believe the receptors may have for example at least 30%, such as at least 40%, at least 50%, at least 60%, and in particular at least 70%, such as at least 80%, for example 85%, or 90% or 95% peptide sequence identity. It is appreciated that homologous receptors may have substantially higher peptide sequence identity over small regions representing functional domains. We include receptors having greater diversity in their DNA coding sequences than outlined for the above amino acid sequences but which give rise to receptors having peptide sequence identity falling within the above sequence ranges. Convenient versions of the LXRα receptor include the published sequence. The amino acid sequence of human LXRα can be obtained from the SwissProt database, accession no Q13133 (NRH3_HUMAN) and the cDNA sequence e.g. from the EMBL database accession no. U22662. The LXRα receptor is from any mammalian species, including human, monkey, rat, mouse and dog. Preferably the human LXRα receptor is used.

Fragments and partial sequences of the LXRα receptor may be useful substrates in the assay and analytical methods of the invention. It will be appreciated that the only limitation on these is practical, they must comprise the necessary functional elements for use in the relevant assay and/or analytical procedures.

The agent of this invention may be administered in standard manner for the condition that it is desired to treat, for example by oral, topical, parenteral, buccal, nasal, or rectal administration or by inhalation. For these purposes the compounds of this invention may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

Knowledge of the LXRα receptor also provides the ability to regulate its expression in vivo by for example the use of antisense DNA or RNA. Thus, according to a further aspect of the invention we provide an appetite control agent comprising an antisense DNA or an antisense RNA which is complementary to all or a part of a polynucleotide sequences shown in sequence nos. 1,3 and 5. By complementary we mean that the two molecules can hybridise to form a double stranded molecule through nucleotide base pair interactions to the exclusion of other molecular interactions.

The antisense DNA or RNA for co-operation with polynucleotide sequence corresponding to all or a part of a LXRα gene can be produced using conventional means, by standard molecular biology and/or by chemical synthesis. The antisense DNA or RNA can be complementary to the full length LXRα receptor gene of the invention or to a fragment thereof. Antisense molecules which comprise oligomers in the range from about 12 to about 30 nucleotides which are complementary to the regions of the gene which are proximal to, or include, the protein coding region, or a portion thereof, are preferred embodiments of the invention. If desired, the antisense DNA or antisense RNA may be chemically modified so as to prevent degradation in vivo or to facilitate passage through a cell membrane and/or a substance capable of inactivating mRNA, for example ribozyme, may be linked thereto and the invention extends to such constructs.

Oligonucleotides which comprise sequences complementary to and hybridizable to the LXRα receptor are contemplated for therapeutic use. U.S. Pat. No. 5,639,595, Identification of Novel Drugs and Reagents, issued Jun. 17, 1997, wherein methods of identifying oligonucleotide sequences that display in vivo activity are thoroughly described, is herein incorporated by reference.

Nucleotide sequences that are complementary to the LXRα receptor encoding nucleic acid sequence can be synthesised for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, Hybrid Oligonucleotide Phosphorothioates, issued Jul. 29, 1997, and U.S. Pat. No. 5,652,356, Inverted Chimeric and Hybrid Oligonucleotides, issued Jul. 29, 1997, which describe the synthesis and effect of physiologically-stable antisense molecules, are incorporated by reference. LXRα gene antisense molecules may be introduced into cells by microinjection, liposome encapsulation or by expression from vectors harbouring the antisense sequence.

Transgenic animal technology is also contemplated, providing new experimental models, useful for evaluating the effects of test compounds on the control of dyslipidemia, insulin resistance syndrome, type 2 diabetes, obesity and eating disorders. LXRα may be deleted, inactivated or modified using standard procedures as outlined briefly below and as described for example in “Gene Targeting; A Practical Approach”, IRL Press, 1993. The target gene or a portion of it, for example homologous sequences flanking the coding region, is preferably cloned into a vector with a selection marker (such as Neo) inserted into the gene to disrupt its function. The vector is linearised then transformed (usually by electroporation) into embryonic stem cells (ES) cells (eg derived from a 129/Ola strain of mouse) and thereafter homologous recombination events take place in a proportion of the stem cells. The stem cells containing the gene disruption are expanded and injected into a blastocyst (such as for example from a C57BL/6J mouse) and implanted into a foster mother for development. Chimaeric offspring may be identified by coat colour markers. Chimeras are bred to ascertain the contribution of the ES cells to the germ line by mating to mice with genetic markers which allow a distinction to be made between ES derived and host blastocyst derived gametes. Half of the ES cell derived gametes will carry the gene modification. Offspring are screened (for example by Southern blotting) to identify those with a gene disruption (about 50% of the progeny). These selected offspring will be heterozygous and may therefore be bred with another heterozygote to produce homozygous offspring (about 25% of the progeny).

Transgenic animals with a target gene deletion (“knockouts”) may be crossed with transgenic animals produced by known techniques such as microinjection of DNA into pronuclei, spheroplast fusion or lipid mediated transfection of ES cells to yield transgenic animals with an endogenous gene knockout and a foreign gene replacement. ES cells containing a targeted gene disruption may be further modified by transforming with the target gene sequence containing a specific alteration. Following homologous recombination the altered gene is introduced into the genome. These embryonic stem cells may subsequently be used to create transgenics as described above. Suitable methods are described in WO 00/34461 University of Texas.

The transgenic animals will display a phenotype, which reflects the role of LXRα in the control of appetite and obesity and will thus provide useful experimental models in which to evaluate the effects of test compounds. Therefore in a further aspect of the invention we provide transgenic animals in which LXRα is deleted, inactivated or modified, and used in evaluating the effects of test compounds in dyslipidemia, insulin resistance syndrome, type 2 diabetes, appetite control and obesity. The LXRα receptor may also be used as the basis for diagnosis, for example to determine expression levels in a human subject, by for example direct DNA sequence comparison or DNA/RNA hybridisation assays. Diagnostic assays may involve the use of nucleic acid amplification technology such as PCR and in particular the Amplification Refractory Mutation System (ARMS) as claimed in our European Patent No. 0 332 435. Such assays may be used to determine allelic variants of the gene, for example insertions, deletions and/or mutations such as one or more point mutations. Such variants may be heterozygous or homozygous. Other approaches have been used to identify mutations in genes encoding similar molecules in obese patients (Yeo et al., 1998, Nature Genetics, 20, 111-112).

In a further aspect of the invention the LXRα receptor can be genetically engineered in such a way that its interactions with other intracellular and membrane associated proteins are maintained but its effector function and biological activity are removed. The genetically modified protein is known as a dominant negative mutant. Overexpression of the dominant negative mutant in an appropriate cell type down regulates the effect of the endogenous protein, thus revealing the biological role of the genes in dyslipidemia, insulin resistance syndrome, type 2 diabetes.

Similarly, the LXRα receptor may also be genetically engineered in such a way that its effector function and biological activity are enhanced. The resultant overactive protein is known as dominant positive mutant. Overexpression of a dominant positive mutant in an appropriate cell type amplifies the biological response of the endogenous, native protein, spotlighting its role in dyslipidemia, insulin resistance syndrome, type 2 diabetes. This also has utility in a screen for detecting anatgonists of the constitutively active receptor in the absence of a ligand.

Therefore, in a further aspect of the invention we provide dominant negative and dominant positive mutants of a LXRα receptor and their use in evaluating the biological role of the LXRα receptor in the control of insulin resistance syndrome, dyslipidemia or type 2 diabetes.

The invention will now be illustrated but not limited by reference to the following specific description and sequence listing [Many of the specific techniques used are detailed in standard molecular biology textbooks such as Sambrook, Fritsch & Maniatis, Molecular cloning, a Laboratory Manual, Second Edition, 1989, Cold Spring Harbor Laboratory Press. Consequently references to this will be made at the appropriate points in the text.]:

### EXAMPLES

### The Effect of PPARγ Activators on LXRα Expression in 3T3-L1 Adipocytes

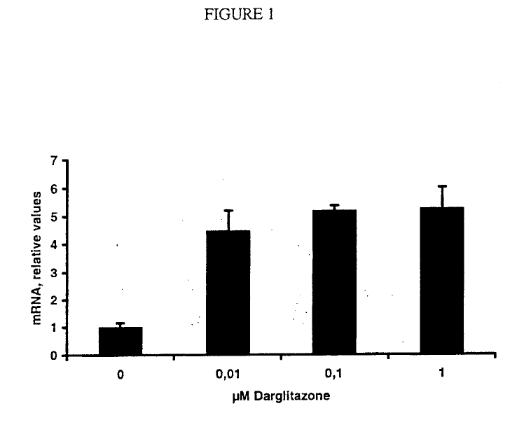

We performed Northern blot analysis on total RNA from 3T3-L1 adipocytes treated with increasing doses of a PPARγ agonist. Adipocytes treated over a 24 hrs period with 0.01, 0.1, and 1 mM of Darglitazone. 20 μg total RNA was subjected to Northern blotting and probed with a ³²P-labeled LXRα cDNA probe. The signal was obtained by scanning the autoradiogram and normalised for 18S RNA expression. Results showed an approximately 5-fold induction of LXRa mRNA (FIG. 1). These concentrations are in agreement with concentrations required for activation of PPARγ in reporter assays (Lehmann et al. 1995). Hence, treatment of adipocytes with a selective PPARγ agonist increases LXRα mRNA levels.

### Treatment of 3T3-L1 Adipocytes with 22-R-Hydroxy Cholesterol and Darglitazone

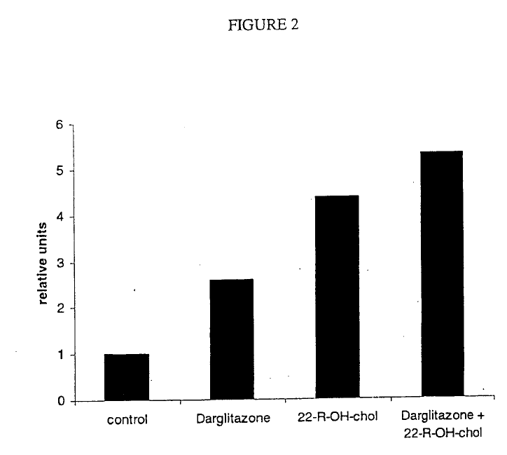

3T3-L1 cells committed to adipocyte differentiation were treated with either Darglitazone, the LXRα agonist 22-R-hydroxy cholesterol, or both. 22-R-hydroxy cholesterol (22-R) is a naturally occurring agonist for LXRα (Janowski et al. 1996). Cells stimulated with 22-R, Darglitazone or both were forming gradually larger lipid droplets, as shown by Oil Red-O staining of the cells. These results indicate that Darglitazone stimulation of PPARγ as well as 22-R stimulation of LXRα leads to increased storage of triglycerides in adipocytes. In parallel, Northern blot analysis of total RNA shows an increase of LXRα mRNA in differentiating 3T3-L1 cells treated with Darglitazone or 22-R and an additive effect by stimulation with both Darglitazone and 22-R (FIG. 2). Therefore, treatment of 3T3-L1 pre-adipocytes with either a PPARγ agonist or an LXRα agonist leads to fat accumulation and increased expression of LXRα.

### Treatment of Human Adipocytes with 22-R-Hydroxy Cholesterol

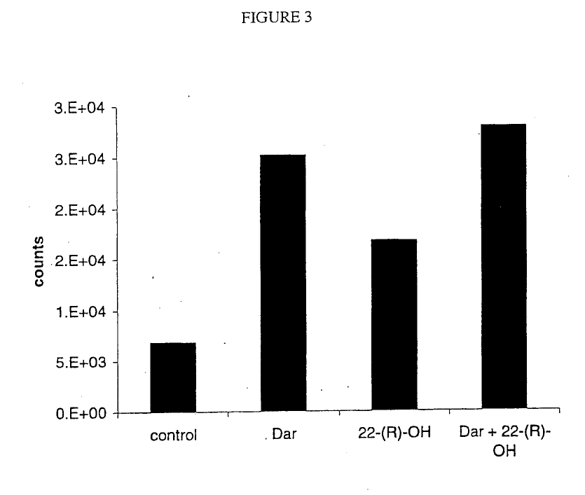

Human adipocytes were obtained from breast reduction surgery. Pieces of adipose tissue (5-600 mg) were prepared under sterile conditions and used for incubations in plastic tubes essentially as described (Ottosson et al., 1994). 1 μM Darglitazone, 5 μM 22-(R)-hydroxy cholesterol or both was added for 48 hrs as indicated in the figure legends. PolyA+ RNA was isolated and subjected to Northern blot analysis (FIG. 3). Both Darglitazone and 22-(R)-hydroxy cholesterol treatment led to increased expression levels of LXRα mRNA with an additive effect. Hence, stimulation with a selective PPARγ agonist or an LXRα agonist leads to upregulation of LXRα mRNA in human adipocytes.

### Cells and Reagent

The 3T3-L1 cell line (ATCC) was maintained in DMEM supplemented with 10% fetal calf serum, 2 mM L-glutamine and penicillin/streptomycin at 37° C. Cells were grown to confluence and exposed to adipogenic reagents for 3 days, followed by culturing for 3 more days in medium containing insulin only as described elsewhere (Lin and Lane, 1992). Insulin was used at a concentration of 1 μg/ml, dexamethasone at 1 μM and isobutylmethylxanthine at 0.5 mM.

### Preparation and Analysis of RNA

Total RNA from differentiated 3T3-L1 adipocytes or adipose tissue were extracted by the Trizol (Life Technologies, Inc.) method as recommended by the manufacturer. Northern blot analysis of RNA was performed as described earlier (Sorensen et al., 1994). 20 μg of total RNA was analyzed for LXRα and ribosomal protein 18S mRNA.

### Oil Red O Staining

Light microscopy and Oil Red O staining were used to monitor the characteristic cell rounding and lipid droplet accumulation in these cells during differentiation. Images were taken using a microscope (Leica DMIL) and a dual-colour charge coupled device camera (Leica MPS 60).

1. A method of stimulating pre-adipocyte differentiation in a cell comprising administering a LXRα agonist to the cell, wherein the agonist stimulates pre-adipocyte differentiation.

2. The method of claim 1, wherein the cell is a mammalian cell.

3. The method of claim 1, wherein the cell is an adipocyte cell, a 3T3-L1 pre-adipocyte cell, or a 3T3-L1 adipocyte cell.

4. The method of claim 1, wherein the LXRα agonist is an oxidized derivative of cholesterol.

5. The method of claim 4, wherein the derivative is selected from the group consisting of 22(R)-hydroxycholesterol, 24(S)-hydroxycholesterol, and 24,25(S)-epoxycholesterol.

6. The method of claim 1, wherein the LXRα agonist is a thiazolidinedione compound.

7. The method of claim 6, wherein the thiazolidinedione compound is selected from the group consisting of darglitazone, rosiglitazone, pioglitazone, or troglitazone, and their pharmaceutically acceptable salts.

8. A method of treating a disorder associated with aberrant pre-adipocyte differentiation, comprising administering a therapeutically effective amount of a LXRα modulator to a mammal, wherein the LXRα modulator stimulates pre-adipocyte differentiation.

9. The method of claim 8, wherein the LXRα modulator is an oxidized derivative of cholesterol.

10. The method of claim 9, wherein the derivative is selected from the group consisting of 22(R)-hydroxycholesterol, 24(S)-hydroxycholesterol, and 24,25(S)-epoxycholesterol.

11. The method of claim 8, wherein the LXRα modulator is a thiazolidinedione compound.

12. The method of claim 11, wherein the thiazolidinedione compound is selected from the group consisting of darglitazone, rosiglitazone, pioglitazone, or troglitazone, and their pharmaceutically acceptable salts.

13. The method of claim 8, wherein the disorder is insulin resistance syndrome, dyslipidemia or type 2 diabetes.

14. The method of claim 8, wherein the LXRα modulator is administered to the mammal in a pharmaceutical composition comprising a pharmaceutically acceptable carrier or excipient.

15-42. (canceled)

43. The method of claim 8, wherein the LXRα modulator is administered orally, topically, intravenously, transdermally, rectally, or parenterally.
